(19) United States
(12) Patent Application Publication
(10) Pub. No.: US 2025/0352710 A1
(43) Pub. Date: Nov. 20, 2025

(54) Systems for Medical Fluid Pumps and Related Methods

(71) Applicant: Fresenius Medical Care Holdings, Inc., Waltham, MA (US)

(72) Inventors: Shu Yun Wang, Malden, MA (US); Ping Huang, Libertyville, IL (US); David Yuds, Hudson, NH (US)

(21) Appl. No.: 18/665,227

(22) Filed: May 15, 2024

(51) Int. Cl. A61M 1/28; A61M 1/00

(52) U.S. Cl. CPC A61M 1/28; A61M 1/69; A61M 1/80; A61M 2205/0294; A61M 2205/3331; A61M 2209/08; A61M 2210/1017

(57) ABSTRACT

This disclosure relates to systems for medical fluid pumps and related methods. In some implementations, a system includes a system for draining fluid from a peritoneal cavity of a patient comprising a microfluidic pump; a drain bag fluidly coupled to the microfluidic pump; and an inlet line coupled to the drain bag, wherein the microfluidic pump is configured to apply a negative pressure to an interior of the drain bag to draw effluent from a peritoneal cavity of the patient along the inlet line and into the drain bag. In some implementations, a system for performing peritoneal dialysis includes a microfluidic pump; a dual chamber bag fluidly coupled to the microfluidic pump, where the dual chamber bag includes an effluent chamber configured to be fluidly coupled to a peritoneal cavity of a patient; a dialysate chamber; and a flexible membrane separating the effluent chamber from the dialysate chamber.

### TECHNICAL FIELD

This disclosure relates to systems for medical fluid pumps and related methods.

### BACKGROUND

Dialysis is a treatment used to support a patient with insufficient renal function. The two principal dialysis methods are hemodialysis and peritoneal dialysis. During peritoneal dialysis (“PD”), a patient's peritoneal cavity is periodically infused with dialysate. The membranous lining of the patient's peritoneum acts as a natural semi-permeable membrane that allows diffusion and osmosis exchanges to take place between the solution and the blood stream. These exchanges across the patient's peritoneum, like the continuous exchange across the dialyzer in HD, result in the removal of waste products, including solutes like urea and creatinine, from the blood, and regulate the levels of other substances, such as sodium and water, in the blood.

Many PD machines are designed to automatically infuse, dwell, and drain dialysate to and from the patient's peritoneal cavity. The treatment typically lasts for several hours, often beginning with an initial drain cycle to empty the peritoneal cavity of used or spent dialysate. The sequence then proceeds through the succession of fill, dwell, and drain phases that follow one after the other. Each phase is called a cycle. Continuous ambulatory peritoneal dialysis (CAPD) therapy involves hanging a bag of fresh dialysate and using gravity to fill a patient's peritoneal cavity. At the end of the dwell phase of the treatment cycle, the patient drains effluent (spent dialysate) from the patient's peritoneal cavity into a drain bag via gravity.

### SUMMARY

In one aspect, a system for draining fluid from a peritoneal cavity of a patient includes a microfluidic pump, a drain bag fluidly coupled to the microfluidic pump, and an inlet line coupled to the drain bag, where the microfluidic pump is configured to apply a negative pressure to an interior of the drain bag to draw effluent from a peritoneal cavity of the patient along the inlet line and into the drain bag. Implementations can include one or more of the following features in any combination.

In some implementations, the microfluidic pump is a piezoelectric pump. In some implementations, a drain line is coupled to the drain bag; and a flow sensor is coupled to the drain line, where the flow sensor is configured to measure the volume a fluid flowing from the drain bag along the drain line. In some implementations, the microfluidic pump is configured to apply a positive pressure to the interior of the drain bag prior to applying the negative pressure to the interior of the drain bag. In some implementations, a connector line is fluidly coupled to the microfluidic pump and the drain bag. In some implementations, a solenoid valve coupled to the connector line and configured to control a flow of fluid generated by the microfluidic pump to the drain bag or away from the drain bag; and a pressure sensor coupled to the connector line, where the pressure sensor is configured to detect a pressure generated by the microfluidic pump and applied to the interior of the drain bag. In some implementations, a casing surrounds the microfluid pump. In some implementations, a control unit is positioned within the casing, the control unit comprising at least one processor configured to control the operations of the microfluidic pump. In some implementations, a valve is coupled to the inlet line, where the valve is configured to control the flow of spent medical fluid into the drain bag. In some implementations, a rigid container is fluidly coupled to the microfluidic pump. In some implementations, a dialysate bag is positioned inside the rigid container.

In a further aspect, a method for performing peritoneal dialysis includes actuating a microfluidic pump to apply a positive pressure to an interior of a drain bag; and actuating the microfluidic pump to apply a negative pressure to the interior of the drain bag to generate a vacuum that causes effluent to flow from a peritoneal cavity of a patient into the drain bag. In some implementations, the method includes flowing effluent from the interior of the drain bag along a drain line coupled to the drain bag; and measuring, with a flow sensor, a volume of the effluent flowed from the drain bag along the drain line with the flow sensor. In some implementations, the method includes actuating the microfluidic pump to apply the positive pressure to a dialysate bag positioned inside an interior of a rigid container fluidly coupled to the microfluidic pump; and opening a valve along a second fluid line fluidly coupling the dialysate bag and the peritoneal cavity of a patient, where a pressure applied to the dialysate bag causes fresh dialysate to flow from the dialysate bag to the patient's peritoneal cavity along the second fluid line. In some implementations, the microfluidic pump is a piezoelectric pump and (a) further includes applying an electric current to the piezoelectric pump generating a vibration at a first frequency, where the first frequency causes air movement from the piezoelectric pump and into the interior of the drain bag. In some implementations, the method includes applying another electric current to the piezoelectric pump generating another vibration at a second frequency, where the second frequency causes air movement from the interior of the drain bag and into the piezoelectric pump. In some implementations, the method includes detecting, with a pressure sensor, the positive pressure of the interior of the drain bag. In some implementations, the method further includes detecting, with a pressure sensor, the negative pressure of the interior of the drain bag.

In a further implementation, a system for administering and draining fluid from a peritoneal cavity of a patient includes a microfluidic pump; a drain bag fluidly coupled to the microfluidic pump, a rigid container fluidly coupled to the microfluidic pump; and a dialysate bag positioned inside the rigid container. In some implementations, the microfluidic pump is a piezoelectric pump. In some implementations, the system further includes an inlet line configured to fluidly couple the drain bag to the peritoneal cavity of the patient; and an outlet line configured to fluidly couple the dialysate bag to the peritoneal cavity of the patient. In some implementations, an inlet valve coupled to the inlet line and configured to control the flow of effluent the inlet line; and an outlet valve coupled to the outlet line and configured to control flow of fresh dialysate along the outlet line. In some implementations, the system further includes a connector line fluidly coupled to the microfluidic pump, the drain bag, and the rigid container; a solenoid valve coupled to the connector line configured to control a flow of fluid generated by the microfluidic pump along the connector line; and a pressure sensor coupled to the connector line and configured to detect a pressure generated by the microfluidic pump. In some implementations, the system further includes a first fluid valve coupled to a first fluid line, and configured to control a flow of fluid generated by the microfluidic pump through the first fluid line; and a second fluid valve coupled to a second fluid line and configured to control the flow of fluid generated by the microfluidic pump through the second fluid line. In some implementations, the system further includes a connector line coupled to the microfluidic pump; a T-connector coupled to the connector line; a first fluid line coupled to the T-connector; and a second fluid line coupled to the T-connector, where the first fluid line and the second fluid line selectively guide a fluid generated by the microfluidic pump. In some implementations, the microfluidic pump is configured to apply a negative pressure to an interior of the drain bag to draw spent medical fluid from the patient to the interior of the drain bag. In some implementations, the system further includes the microfluidic pump is configured to apply a positive pressure to an interior of the rigid container to thereby affect a pressure to an external portion of the dialysate bag.

In a further aspect, a method of performing peritoneal dialysis includes actuating a microfluidic pump to apply a positive pressure to an interior of a drain bag fluidly coupled to the microfluidic pump; actuating the microfluidic pump to apply a negative pressure to the interior of the drain bag to generate a vacuum inside the drain bag; opening a first valve along a first fluid line fluidly coupling the drain bag and a peritoneal cavity of a patient, where the vacuum generated inside the drain bag causes effluent to flow from the patient's peritoneal cavity to the drain bag; actuating the microfluidic pump to apply the positive pressure to a dialysate bag positioned inside an interior of a rigid container fluidly coupled to the microfluidic pump; and opening a second valve along a second fluid line fluidly coupling the dialysate bag and the peritoneal cavity of the patient, where the positive pressure applied to the dialysate bag causes fresh dialysate to flow from the dialysate bag to the patient's peritoneal cavity along the second fluid line.

In some implementations, the method further includes flowing effluent from the drain bag along a drain line coupled to the drain bag; and measuring, with a flow sensor, a volume of the effluent flowing from the drain bag along the drain line with the flow sensor In some implementations, the microfluidic pump is a piezoelectric pump and actuating the microfluidic pump to apply the positive pressure to an interior of the drain bag includes applying an electric current to the piezoelectric pump to generate a vibration at a first frequency, where the first frequency causes air movement from the piezoelectric pump into the interior of the drain bag. In some implementations, actuating the microfluidic pump to apply the negative pressure to the interior of the drain bag includes applying a second electric current to the piezoelectric pump to generate a second vibration at a second frequency, where the second frequency causes air movement from the interior of the drain bag into the piezoelectric pump.

In some implementations, a system for performing peritoneal dialysis includes a microfluidic pump; a dual chamber bag fluidly coupled to the microfluidic pump, where the dual chamber bag includes an effluent chamber configured to be fluidly coupled to a peritoneal cavity of a patient and receive effluent from the peritoneal cavity of the patient; a dialysate chamber configured to be fluidly coupled to the peritoneal cavity of a patient and to contain dialysate; and a flexible membrane separating the effluent chamber from the dialysate chamber. In some implementations, the microfluidic pump is a piezoelectric pump. In some implementations, the microfluidic pump is configured to apply a negative pressure to an interior of the effluent chamber to draw spent medical fluid from the patient to the interior of the effluent chamber. In some implementations, the flexible membrane is configured to flex to apply a pressure to the dialysate chamber when the effluent chamber is filled with effluent.

In some implementations, the pressure to the dialysate chamber is from a weight of the effluent contained in the effluent chamber. In some implementations, the system further includes an inlet line coupled to the effluent chamber; an inlet valve coupled to the inlet line, where the inlet valve is configured to control flow of effluent into the effluent chamber from the peritoneal cavity of the patient; an outlet line coupled to the dialysate chamber; and an outlet valve coupled to the outlet line, where the outlet valve is configured to control flow of dialysate from the dialysate chamber to the peritoneal cavity of the patient. In some implementations, the system further includes a rigid case configured to contain the microfluidic pump and the dual chamber bag. In some implementations, the rigid case includes a heater configured to heat dialysate contained in the dialysate chamber. In some implementations, the rigid case further includes a handle and wheels.

In a further aspect, a method of performing peritoneal dialysis treatment includes actuating a microfluidic pump to apply a positive pressure to an interior of an effluent chamber of a dual chamber bag fluidly coupled to the microfluidic pump; actuating the microfluidic pump to apply a negative pressure to the interior of the effluent chamber of the dual chamber bag, where the negative pressure in the interior of the effluent chamber creates a vacuum inside the effluent chamber; flowing effluent from a peritoneal cavity of a patient into the effluent chamber;
applying pressure to a dialysate chamber of the dual chamber bag, the dialysate chamber comprising dialysate; and flowing the dialysate from the dialysate chamber into the patient's peritoneal cavity. In some implementations, flowing effluent from the peritoneal cavity of the patient into the effluent chamber includes opening a valve along an inlet line fluidly coupling the effluent chamber to the peritoneal cavity. In some implementations, flowing the dialysate from the dialysate chamber into the patient's peritoneal cavity includes opening a valve along an outlet line fluidly coupling the effluent chamber to the peritoneal cavity. In some implementations, the method further includes positioning the dual chamber bag inside a rigid case comprising a handle and one or more wheels; and transporting the dual chamber bag during the treatment.

Implementations can include one or more of the following advantages.

In some implementations, the integration of a piezoelectric disc pump implements ultrasonic vibrations to mobilize fluid (e.g., air), which in turn directs flow of the medical fluid (spent dialysate and fresh dialysate). This is advantageous because the mechanism offers a silent operation and enhances the system's reliability in fluid management.

In some implementations, the PD systems and methods disclosed herein use a closed-loop framework comprising valves, a pressure sensor, and a flow sensor that can work in tandem to perpetually monitor and modify the flow and pressure of the medical fluid. This surveillance ensures accurate control over the medical fluid dynamics.

In some implementations, the pressure-driven flow avoids direct liquid pumping. Instead, the PD systems and methods disclosed herein employs air pressure, which is generated by drawing air into a reservoir to propel the fluid. This approach circumvents the need for direct contact with the medical fluid, thus minimizing wear, minimizing potential exposure to contaminants, and extending the PD system's life.

In some implementations, the PD system disclosed herein include a non-contact flow meter configured to measure the flow rate of the medical fluid without making physical contact. This can be advantageous by avoiding problems such as pressure loss and clogging, ensuring consistent performance regardless of the medical fluid characteristics.

In some implementations, the PD systems and methods disclosed herein improve the efficiency and cost-effectiveness by its streamlined design. By eliminating the bulky, noisy, and expensive components characteristic of conventional pumps or scales, the systems described herein are rendered more compact, simple, and cost-efficient.

In some implementations, the design of the PD systems and methods disclosed herein allow for indirect contact with the medical fluid, as the microfluidic pump exerts pressure on a container or bag holding the medical fluid. This separation significantly reduces the risk of contamination, promoting a more hygienic environment.

In some implementations, the PD systems and methods disclosed herein allow for adaptability demonstrated through its disposable components, such as the microfluidic pump, the tubing, valves, and/or dual chamber bag. This advantage simplifies maintenance and enhances hygiene and convenience, making the system an exemplary choice for PD treatments.

In some implementations, the PD systems and methods disclosed herein provide a compact solution that can provide mobility options such that the patient can move around while administering a PD treatment. Such advantages can improve a quality of life by increasing the circumstances under which a patient can receive a PD treatment.

In some implementations, the PD systems and methods disclosed herein allow for flexibility in the configuration of the drain bag and/or the dialysate bag. For example, the drain bag is not dependent on gravity to receive effluent from the patient can be placed on a table as the PD systems and methods use positive and negative pressure to direct the flow of fluid. In another example, the dialysate bag is not dependent on gravity to provide dialysate fluid to the peritoneal cavity of a patient due to positive pressure applied to a rigid container surrounding the dialysate bag. Such advantages can improve a quality of life by increasing the circumstances under which a patient can configure their PD treatment.

The details of certain implementations are set forth in the accompanying drawings and the description below. Other aspects, features, and advantages will be apparent from the description and drawings, and from the claims.

### DESCRIPTION OF DRAWINGS

FIG. 1 is an illustration of a patient receiving peritoneal dialysis (PD) treatment using an example PD treatment system.

FIGS. 2A-2C depict a patient receiving PD treatment using another example PD treatment system.

FIG. 3 is an illustration of another example PD treatment system.

FIGS. 4A-4C depict an example process of performing PD treatment using the example PD system of FIG. 3.

FIG. 5 is a flowchart showing a method of administering a PD treatment using a microfluidic pump coupled to a drain bag of FIG. 1.

FIG. 6 is a flowchart showing a method of administering a PD treatment using the example PD treatment system of FIGS. 2A-2C.

FIG. 7 is a flowchart showing a method of administering a PD treatment using the example PD treatment system of FIG. 3.

Like reference symbols in the various drawings indicate like elements.

### DETAILED DESCRIPTION

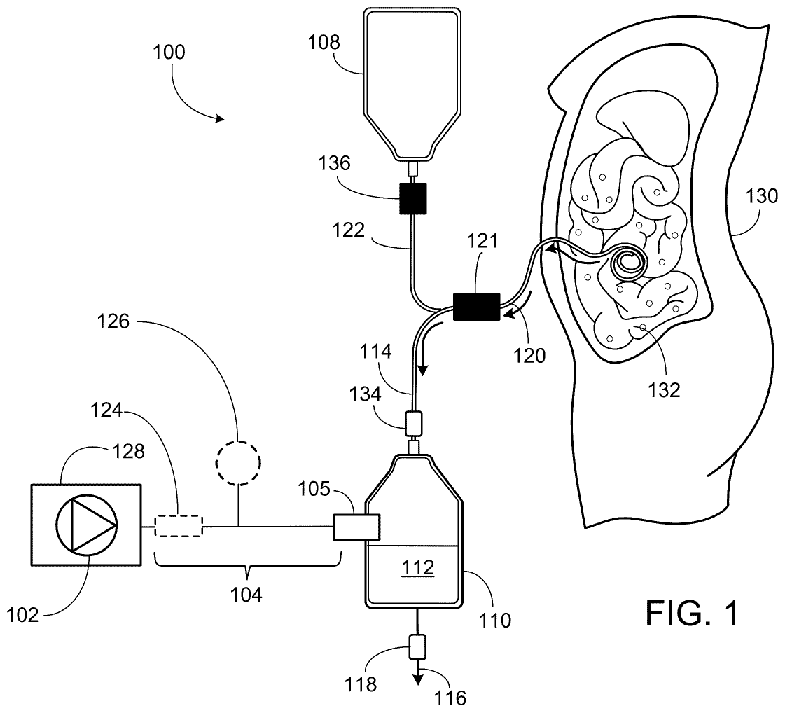

FIG. 1 depicts a patient 130 receiving a peritoneal dialysis (“PD”) treatment using an example PD system 100. The PD system 100 includes a microfluidic pump 102, a dialysate bag 108, and a drain bag 110. The microfluidic pump 102 is fluidically connected to the drain bag 110 by a connector line 104. The drain bag 110 is connected to an inlet line 114 and to a transfer set 120 that is connected to the patient 130 (e.g., a patient catheter). The drain bag 110 is coupled to a drain line 116. The PD system 100 includes a dialysate bag 108 that is coupled to the patient 130 via an outlet line 122 and the transfer set 120.

The PD system 100 of FIG. 1 can be used to perform a continuous ambulatory peritoneal dialysis (CAPD) treatment. CAPD treatment typically begins by draining medical fluid (e.g., spent medical fluid) from a patient's peritoneal cavity 132. Once the patient's peritoneal cavity has been drained, the patient's peritoneal cavity is filled with fresh medical fluid (e.g., dialysate) contained inside the dialysate bag 108, which then dwells in the patient's peritoneal cavity. After delivering the fresh dialysate to the patient's peritoneal cavity 132 and permitting the dialysate to dwell in the peritoneal cavity 132 for a predetermined period of time, the spent medical fluid (e.g., effluent) is drained from the peritoneal cavity 132. These processes of draining, filling, dwelling, and draining is repeated throughout a CAPD treatment cycle.

In order to drain the effluent from the patient's peritoneal cavity 132, the PD system 100 includes a drain bag 110 that is fluidly connected to the patient's peritoneal catheter using a transfer set 120 coupled to the inlet line 114. The transfer set 120 is coupled to a transfer set valve 121 that is configured to selectively permit or prevent medical fluid flow to or from the patient 130 through the transfer set 120. The inlet line 114 includes an inlet valve 134 inlet valve configured to permit effluent 112 from the transfer set 120 to enter the drain bag 110 through the inlet line 114 while preventing the effluent 112 from entering the transfer set 120 from the drain bag 110. During the drain phase of the PD treatment, inlet valve 134 is opened to fluidly couple the drain bag 110 to the patient's transfer set 120 along the inlet line 114, and fluid flows from the peritoneal cavity 132 of the patient into the drain bag 110.

As depicted in FIG. 1, the microfluidic pump 102 is fluidly coupled to the drain bag 110 and is configured to generate a vacuum inside of the drain bag 110 to prompt a drain phase of a PD treatment. For example, to generate a vacuum inside the drain bag 110 during the drain phase of treatment, the microfluidic pump 102 is configured to first apply a positive pressure to the interior of the drain bag 110. Subsequently, at the end of a dwell period of the PD treatment, the microfluidic pump 102 is configured to apply a negative pressure to the interior of the drain bag 110. After the dwell phase, the negative pressure inside the drain bag 110 is generated by the microfluidic pump 102 and the inlet valve 134 along the inlet line 114 is opened, which causes the effluent 112 to flow into the drain bag 110 along the inlet line 114. Applying a positive pressure to the interior of the drain bag 110 allows for a controlled and gradual transition to a vacuum when the microfluidic pump 102 applies a negative pressure to the drain bag 110. The positive pressure inflates the drain bag 110 slightly to provide compressible space for the effluent 112 to flow out of the patient 130 as the microfluidic pump 102 begins applying negative pressure to draw the air out of the drain bag 110. As a result, applying a positive pressure to the drain bag 110 using the microfluidic pump 102 prior to applying the negative pressure helps in more precisely controlling the vacuum generated inside the drain bag 110, which improves patient safety and comfort. Additionally, applying a positive pressure to the interior of the drain bag 110 prevents the drain bag 110 from collapsing when negative pressure is applied. A collapsed drain bag 110 could reduce the effectiveness of the vacuum and disrupt the flow of effluent 112 into the drain bag 110.

The connection of the connector line 104 to the interior of the drain bag 110 produces an airtight seal, which enables the microfluidic pump 202 to provide a controlled positive or negative pressure to the drain bag 110. For example, the connection of the connector line 104 and the drain bag 110 is a luer-lock connector 105 comprising two parts: a male part (the plug) with a conical or tapered shape, and a female part (the socket), which has a corresponding conical or tapered cavity.

The microfluidic pump 102 includes a casing 128. The casing 128 is configured to house electrical components and a power source (e.g., one or more batteries). For example, the casing 128 houses a controller and at least one processor which can be used to record, store, and wirelessly transmit one or more parameters related to the PD treatment performed using the PD system 100.

For example, the casing 128 houses a controller and at least one processor which can be used to record, store, and wirelessly transmit one or more parameters related to the PD treatment, other embodiments are possible. For example, a controller, in conjunction with at least one processor, is configured to execute the operation of the microfluidic pump 102, the valves (e.g., solenoid valve 124, transfer set valve 121, inlet valve 134, and outlet valve 136). In some implementations, the processor executes instructions from the controller to manage the flow rate and direction of flow of fresh and effluent. Further, the processor executes instructions from the controller to manage the flow rate and direction of flow of fluid (e.g., positive air pressure or negative air pressure) generated by the microfluidic pump 102 to the delivery of fresh and effluent as part of the PD treatment.

The microfluidic pump 102 is a piezoelectric disc pump. The microfluidic pump 102 is fluidly coupled to a solenoid valve 124 and a pressure sensor 126 via the connector line 104. When an electric current is applied to the piezoelectric disc pump 102, the disc changes shape (e.g., vibrates) due to a piezoelectric effect. The frequency of these vibrations can be in the ultrasonic range. The piezoelectric disc pump 102 is configured to create a positive pressure by vibrating at a frequency that causes the disc to move air out of a casing 128 through the connector line 104 and to the interior of the drain bag 110. The pressure along the connecter line 104 is monitored by the pressure sensor 126. To create a vacuum, the piezoelectric disc pump 102 draws from the interior of the drain bag 110 and out of the system (e.g., through the casing 128). For example, by adjusting the vibration pattern or frequency of the piezoelectric disc, the microfluidic pump 102 is configured to pull fluid (e.g., air) into the casing 128, generating a negative pressure inside the drain bag 110 thereby generating a vacuum. The pressure ranges generated by the microfluidic pump 102 are between about 6 mmHg to about 13.5 mmHg.

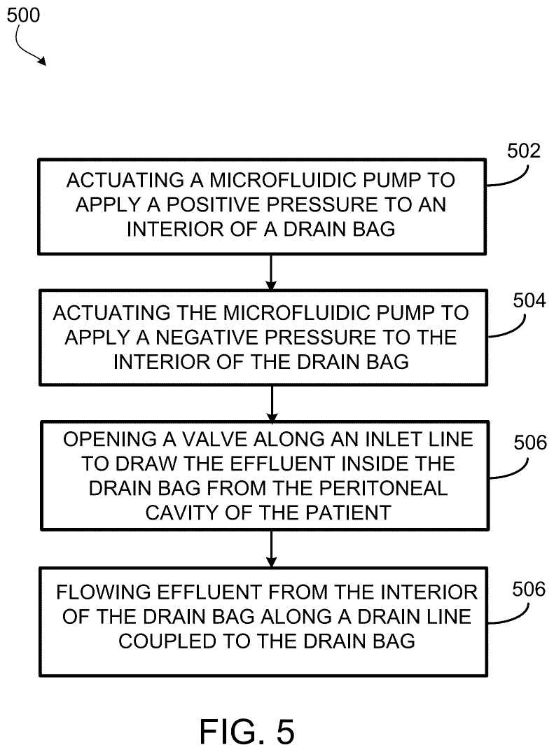

An example process of performing a drain phase of a PD treatment using the example PD system 100 will be described with reference to FIG. 1 and FIG. 5. FIG. 5 is a flowchart showing an example method 500 for draining effluent 112 from the peritoneal cavity 132 of a patient 130 during a PD treatment with the microfluidic pump 102. Prior to performing the drain phase of a PD treatment cycle, the drain bag 110 is connected to the inlet line 114 and to the transfer set 120 that is connected to the patient 130. Once the drain bag 110 is connected to the inlet line 114, a drain phase of a PD treatment begins by opening the solenoid valve 124 along the connector line 104 and actuating the microfluidic pump 102 to apply a positive pressure to the interior of the drain bag 110 (502). As the microfluidic pump 102 actuates, the microfluidic pump 102 pushes fluid (e.g., air) through the connector line 104 and to the interior of the drain bag 110, which applies a positive pressure to the interior of the drain bag 110. The drain bag 110 is connected to the inlet line 114 while the drain bag 110 is filled with fluid from the positive pressure generated by the microfluidic pump 102, and the inlet valve 134 prevents the fluid generated by the positive pressure from entering the transfer set 120.

After applying a positive pressure to the drain bag 110 and at the end of a dwell period of the PD treatment, the microfluidic pump 102 is actuated to apply a negative pressure to the interior of the drain bag 110 by drawing the positive pressure fluid from the interior of the drain bag 110 into the casing 128 (504). The negative pressure generated by the microfluidic pump 102 produces a vacuum in the interior of the drain bag 110. Once the microfluidic pump 102 has generated a vacuum within the drain bag 110, the inlet valve 134 along the inlet line 114 is opened, and the vacuum inside the drain bag 110 draws the effluent 112 to the drain bag 110 (506). In some implementations, a hydrophobic filter is positioned along the connector line 104 (e.g., coupled to connector 105) between the microfluidic pump 102 and the drain bag 110 and the pressure along the connector line 104 is monitored during the drain phase (e.g., using pressure sensor 126) in order to detect a change in pressure along the connector line 104. For example, if the effluent 112 flows from the drain bag 110 into the hydrophobic filter along the connector line 104 as a result of the vacuum pressure generated by the microfluidic pump 102, the pressure within the connector line 104 increases. In some implementations, the microfluidic pump 102 is controlled to stop applying a vacuum to the drain bag 110 in response to detecting the increased pressure along the connector line 104.

Because the drain procedure is not reliant on gravity to flow the effluent from the peritoneal cavity of the patient to the drain bag 110, but rather the pressure generated by the microfluidic pump 202, the drain bag 110 can be placed at or above the height of the patient's peritoneal cavity 232, such as on a table next to the patient, which prevents the patient from having to bend over to pick up the drain bag 110 filled with effluent at the end of the drain phase.

As depicted in FIG. 1, the drain bag 110 is coupled to a drain line 116 and a flow sensor 118. The drain line 116 is configured to direct the effluent 112 from the drain bag 110 into a drain or a waste receptacle. In some implementations, the drain line is used to flow effluent from the interior of the drain bag along the drain line to a drain (508). The flow sensor 118 is configured to monitor a volume of effluent 112 exiting the drain bag 110. As fluid flows out of the drain bag 110, the flow sensor 118 measures the amount of fluid flowing along the drain line 116 in order to measure the amount of spent effluent drained from the patient 130. The flow sensor 118 informs the patient 130 if a sufficient amount of effluent 112 has been drained from their peritoneal cavity 132. For example, the flow sensor 118 is communicatively coupled via a wireless connection to an external computing device (e.g., the patient's phone) and the flow sensor 118 data is monitored to compare the volume of effluent 112 drained against the expected volume based on the treatment parameters. If the volume of effluent 112 drained is less than expected, indicating an incomplete fluid exchange, the flow sensor 118 can trigger an alert (e.g., an audible alarm) to the patient or healthcare provider.

Once the patient's peritoneal cavity 132 has been drained of effluent 112, the fill phase of the PD treatment is performed by connecting the dialysate bag 108 containing fresh dialysate to the transfer set 120 using the outlet line 122 and delivering about 1-3 liters of fresh dialysate from the dialysate bag 108 to the peritoneal cavity 132. In some implementations, the fresh dialysate in the dialysate bag 108 is heated prior to beginning the fill phase.

As depicted in FIG. 1, the dialysate bag 108 is not coupled to the microfluidic pump 102. To fill the patient's peritoneal cavity 132 with fresh dialysate without the use of the microfluidic pump 102, the dialysate bag 108 is positioned above the outlet line 122 and the transfer set 120, which allows for gravity filling of the patient's peritoneal cavity 132. By hanging the dialysate bag 108 on a stand, the fresh dialysate flows downwards via gravity along the outlet line 122 and into the transfer set 120. An outlet valve 136 is positioned along the outlet line 122 to control fresh dialysate flow from the dialysate bag 108 along the outlet line 122. Once the dialysate bag 108 has been attached to a stand and opposite ends of the outlet line 122 are coupled to the dialysate bag 108 and the transfer set 120, the outlet valve 136 positioned along the outlet line 122 is opened to allow fresh dialysate to flow via gravity from the dialysate bag 108. The fresh dialysate flows through the outlet line 122, through the patent line 120, through the patient's catheter, and into the peritoneal cavity 132 of the patient 130.

While certain embodiments have been described, other embodiments are possible.

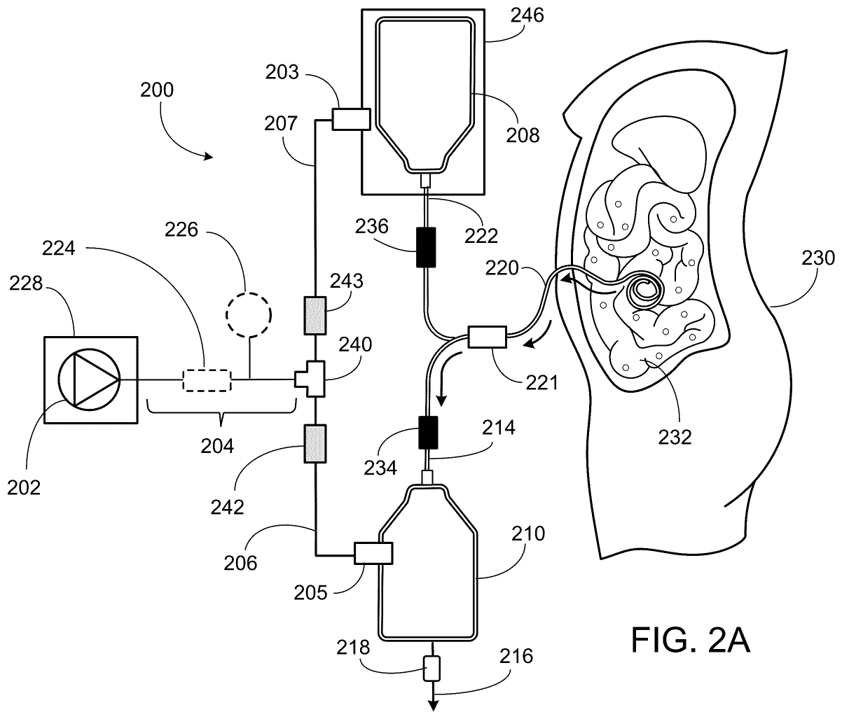

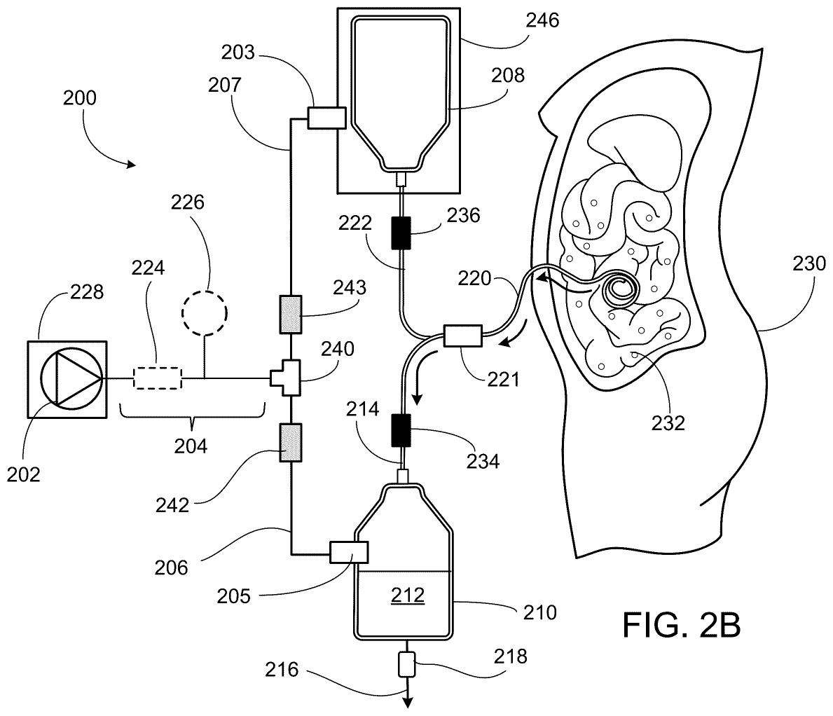

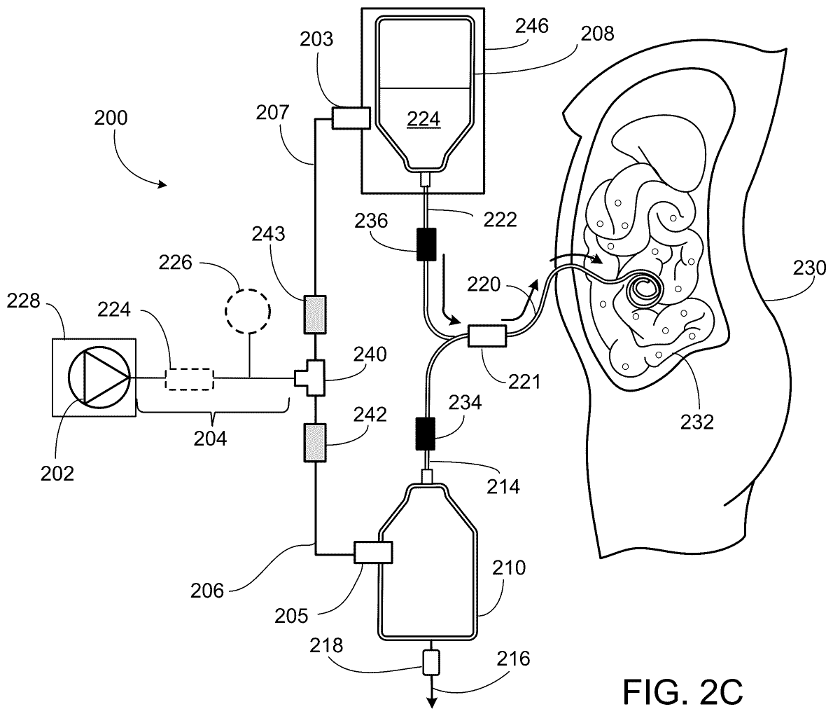

For example, while the microfluidic pump 102 has been depicted as only being operated during the drain phase of PD treatment, in some implementations, the microfluidic pump 102 can be used to facilitate both the drain phase and the fill phase of PD treatment. FIGS. 2A-2C show a schematic of a patient 230 receiving a PD treatment using another example PD system 200 that includes a microfluidic pump 202 for facilitating both the drain phase and the fill phase of the PD treatment. Referring to FIGS. 2A-2C, the PD system 200 includes a microfluidic pump 202, a dialysate bag 208 positioned inside a rigid container 246, and a drain bag 210. A connector line 204 is fluidly coupled to the microfluidic pump 202. A T-connector 240 is coupled to the end of the connector line 204 and fluidically connects the microfluidic pump 202 to the rigid container 246 containing the dialysate bag 208 and to the drain bag 210. The microfluidic pump 202 is fluidically connected to the drain bag 210 by the connector line 204 coupled to the T-connector 240 and a first fluid line 206 fluidly coupled to and extending from the drain bag 210. The microfluidic pump 202 is fluidically connected to the rigid container 246 containing the dialysate bag 208 by a connector line 204 coupled to the T-connector 240 and a second fluid line 207 fluidly coupled to and extending from the rigid container 246.

The casing 228 houses a controller and at least one processor which can be used to record, store, and wirelessly transmit one or more parameters related to the PD treatment, other embodiments are possible. For example, a controller, in conjunction with at least one processor, is configured to execute the operation of the microfluidic pump 202, the valves (e.g., solenoid valve 224, transfer set valve 221, inlet valve 234, and outlet valve 236). In some implementations, the processor executes instructions from the controller to manage the flow rate and direction of flow of fresh and effluent. Further, the processor executes instructions from the controller to manage the flow rate and direction of flow of fluid (e.g., positive air pressure or negative air pressure) generated by the microfluidic pump 302 to the delivery of fresh and effluent as part of the PD treatment.

The microfluidic pump 202 is a piezoelectric disc pump. The microfluidic pump 202 is fluidly coupled to a solenoid valve 224 and a pressure sensor 226 via the connector line 204. When an electric current is applied to the microfluidic pump 202, the disc of the piezoelectric disc pump 202 changes shape (e.g., vibrates) due to a piezoelectric effect. The frequency of these vibrations can be in the ultrasonic range. The piezoelectric disc pump 202 can create a positive pressure by vibrating at a frequency that causes the disc to move air out of a casing 228, through the connector line 204, the T-connector 240, and through the first fluid line 206 or the second fluid line 207. A first fluid valve 242 is positioned along the first fluid line 206. The first fluid valve 242 is configured to selectively permit or prevent fluid generated by the microfluidic pump 202 from entering or exiting the drain bag 210 through the first fluid line 206. Similarly, a second fluid valve 243 is positioned along the second fluid line 207 and is configured to selectively permit or prevent fluid generated by the microfluidic pump 202 from entering or exiting the rigid container 246 through the second fluid line 207. The pressure is monitored by the pressure sensor 226.

The connection of the first fluid line 206 to the interior of the drain bag 210 produces an airtight seal, which enables the microfluidic pump 202 to provide a controlled positive or negative pressure to the drain bag 210. Similarly, the connection of the second fluid line 207 to the interior of the rigid container 246 produces an airtight seal, which enables the microfluidic pump 202 to provide a controlled positive or negative pressure to the rigid container 246 containing the dialysate bag 208. For example, the first fluid line 206 is connected to the drain bag 210 and the second fluid line 207 is connected to the rigid container 246 is using a respective luer-lock connector 205, 203. The luer-lock connectors 205 and 203 each comprise two parts: a male part (the plug) with a conical or tapered shape, and a female part (the socket), which has a corresponding conical or tapered cavity.

The microfluidic pump 202 includes a casing 228. The casing 228 is configured to house electrical components and a power source (e.g., one or more batteries). For example, the casing 228 can house a controller and at least one processor which can be used to record, store, and wirelessly transmit one or more parameters related to the PD treatment.

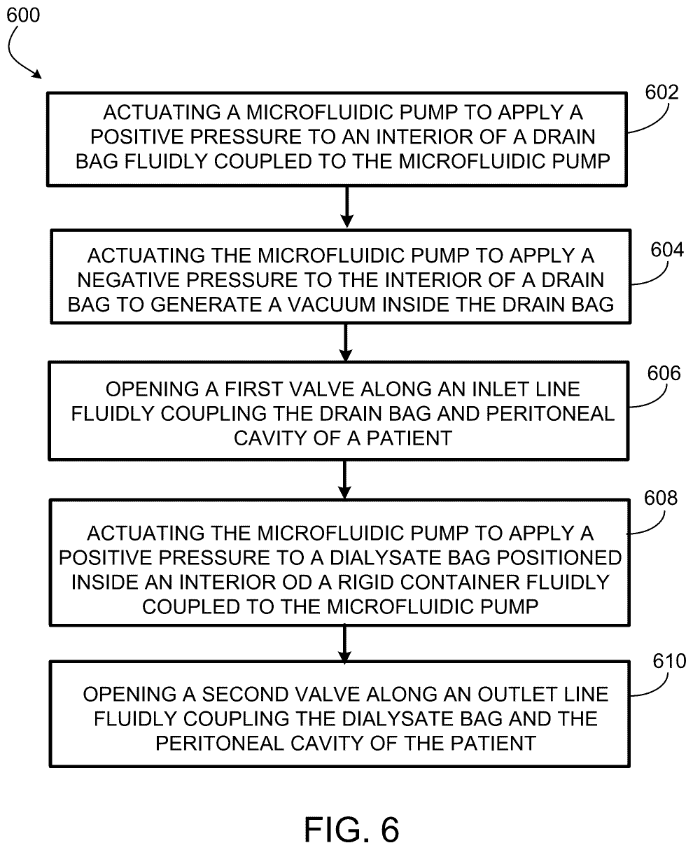

An example process of performing a PD treatment using the example system 200 will now be described with reference to FIGS. 2A-2B and 2C. FIG. 6 is a flowchart showing an example method 600 for performing a PD treatment. Prior to performing the drain phase of a PD treatment cycle, a drain bag 210 is fluidly connected to a microfluidic pump 202 along an inlet line 214 and is fluidly coupled to the peritoneal cavity 232 of a patient 230 along a transfer set 220. Once the drain bag 210 is connected to the inlet line 214, the microfluidic pump 202 is actuated to apply a positive pressure to the interior of the drain bag 210 fluidically coupled to the microfluidic pump 202 via a connector line coupled to the microfluidic pump 202 (602). As discussed above, applying a positive pressure to the interior of the drain bag 210 allows for a controlled and gradual transition to a vacuum when the microfluidic pump 202 applies a negative pressure to the drain bag 210. This helps in controlling the vacuum process more precisely, which can improve patient safety and comfort.

Valves control the flow of fluid (e.g., air) through the system 200. For example, when the valves 234, 242 along the inlet line 214 and the first fluid line 206, respectively, are opened, the valves 236, 243 along the outlet line 222 and second fluid line 207, respectively, are closed. The valve 242 along the first fluid line 206 is opened, and the microfluidic pump 202 actuates to pump fluid (e.g., air) through the connector line 204 and the first fluid line 206 into the interior of the drain bag 210, which generates a positive pressure within the drain bag 210. The drain bag 210 is connected to the inlet line 214 and the interior of the drain bag 210 is filled with fluid pumped by the microfluidic pump 202 along the first fluid line 206 into the interior of the drain bag 210. The inlet valve 234 is closed while the positive pressure is applied to the drain bag 210 and is open when a vacuum is generated within the drain bag 210. The transfer set 220 is coupled to a transfer set valve 221 that is configured to selectively permit or prevent medical fluid flow (e.g., fresh dialysate or effluent) to or from the patient 230 through the transfer set 220.

The microfluidic pump 202 is a piezoelectric pump that is configured to generate a positive pressure by vibrating at a frequency that causes air movement from the piezoelectric pump through the connector line 204 and the T-connector 240 to the first fluid line 206 and into the interior of the drain bag 210. By filling the interior volume of the drain bag 210 with fluid (e.g., air), the microfluidic pump 202 produces a positive pressure within the interior of the drain bag 210. Filling the interior volume of the drain bag 210 with fluid (e.g., air) prepares the drain bag 210 to collect effluent at the end of a dwell period by preventing the drain bag 210 from collapsing when negative pressure is applied to the drain bag 210, as is further described in connection with FIG. 2B.

For example, turning to the embodiment of FIG. 2B, at the end of a dwell period of the PD treatment, the valves 236, 243 along the outlet line 222 and second fluid line 207, respectively, are closed, the valves 242 along first fluid line 206 is opened, and the microfluidic pump 202 actuates to apply a negative pressure to the interior of the drain bag 210 to generate a vacuum inside the drain bag (604). For example, the microfluidic pump 202 actuates to draw the positive pressure fluid from the interior of the drain bag 210 through the connector line 204 and the T-connector 240 and into the casing 228 of the microfluidic pump 202, which produces a vacuum in the interior of the drain bag 210. The microfluidic pump 202 is a piezoelectric pump that is configured to generate a negative pressure by vibrating at a frequency that pulls air movement through the connector line 204, the T-connector 240, and the first fluid line 206 and from the interior of the drain bag 210. The negative pressure (e.g., air vacuum) in the interior of the drain bag 210 that draws the effluent 212 to the drain bag 210. The pressure ranges generated by the microfluidic pump 202 are between about 6 mmHg to about 13.5 mmHg.

Once the microfluidic pump 202 has generated a vacuum inside the drain bag 210, the valve 234 along the inlet line 214 is opened and effluent 212 from the patient's peritoneal cavity 232 is drawn through the inlet line 214 and into the drain bag 210 (606). Because the drain procedure is not reliant on gravity to flow the effluent from the peritoneal cavity of the patient to the drain bag 210, but rather the pressure generated by the microfluidic pump 202, the drain bag 210 can be placed at or above the height of the patient's peritoneal cavity 232, such as on a table next to the patient, which prevents the patient from having to bend over to pick up the drain bag 210 filled with effluent at the end of the drain phase.

As depicted in FIGS. 2A-2C, the drain bag 210 is coupled to a drain line 216 and a flow sensor 218. The drain line 216 is configured to direct the effluent 212 from the drain bag 210 into a drain or a waste receptacle. The flow sensor 218 is configured to monitor a volume of effluent 212 exiting the drain bag 210 along the drain line 216, which can be used to determine the amount of effluent drained from the patient 230. In some implementations, the system 200 is configured to inform the patient 230 if a sufficient amount of effluent 212 has been drained from their peritoneal cavity 232. The flow sensor 218 is communicatively coupled via a wireless connection to a computing device (e.g., a mobile device of the patient) and the flow sensor 218 data is monitored to compare the volume of effluent 212 drained against the expected volume based on the treatment parameters. If the volume of effluent 212 drained is less than expected, indicating an incomplete fluid exchange, the flow sensor 218 can trigger an alert (e.g., an audible alarm) to the patient or healthcare provider. If the volume of effluent 212 drained is sufficient, the patient can be alerted via an audible alarm that the drain phase is complete.

Once the patient's peritoneal cavity 232 has been drained of effluent 212, the fill phase of the PD treatment can be performed. FIG. 2C shows an example fill phase with the dialysate bag 208 containing fresh dialysate 244 and positioned inside of the rigid container 246. To perform a fill phase of the PD treatment using the example system 200, the valves 236, 243 along the outlet line 222 and second fluid line 207, respectively, are opened, the valves 234, 242 along the inlet line 214 and the first fluid line 206, respectively, are closed, and the microfluidic pump 202 is actuated to apply a positive pressure to an interior of the rigid container 246 to pressurize the interior of the rigid container 246, which thereby applies a positive pressure to an external portion of the dialysate bag 208 positioned in the rigid container 246 (608).

As described above, the microfluidic pump 202 is a piezoelectric pump that is configured to generate a positive pressure by vibrating at a frequency that causes air movement from the piezoelectric pump through the connector line 204 and the T-connector 240 to the second fluid line 207 and into the interior of the rigid container 246. The positive pressure (e.g., air) filling the rigid container 246 applies pressure to the external portion of the dialysate bag 208. In particular, applying positive pressure to the interior of the rigid container 246 results in a compressive force on the external surface of the dialysate bag 208. The compressive force reduces the interior volume of the dialysate bag 208, causing the fresh dialysate 244 inside to be displaced and propelled through the outlet line 222.

While applying a positive pressure to the dialysate bag 208, an outlet valve 236 along the outlet line 222 is opened (610) to allow fresh dialysate 244 to flow along the outlet line 222 to flow to the patient's peritoneal cavity. As a result of the positive pressure applied to the external portion of the dialysate bag 208, fresh dialysate inside the dialysate bag 208 is forced to flow through the transfer set 220 via the outlet line 222 delivering about 1-3 liters of fresh dialysate to the peritoneal cavity 132. The positive pressure continues to be applied at a controlled rate until the fresh dialysate 244 has exited the dialysate bag 208. Because the fill procedure is not reliant on gravity to flow the dialysate from the dialysate bag 208 to the patient, but rather the pressure generated by the microfluidic pump 202, the dialysate bag 208 can be placed at or below the height of the patient's peritoneal cavity 232, which prevents the patient from having to raise the full dialysate bag 208 over their head. The pressure ranges generated by the microfluidic pump 302 are between about 6 mmHg to about 13.5 mmHg.

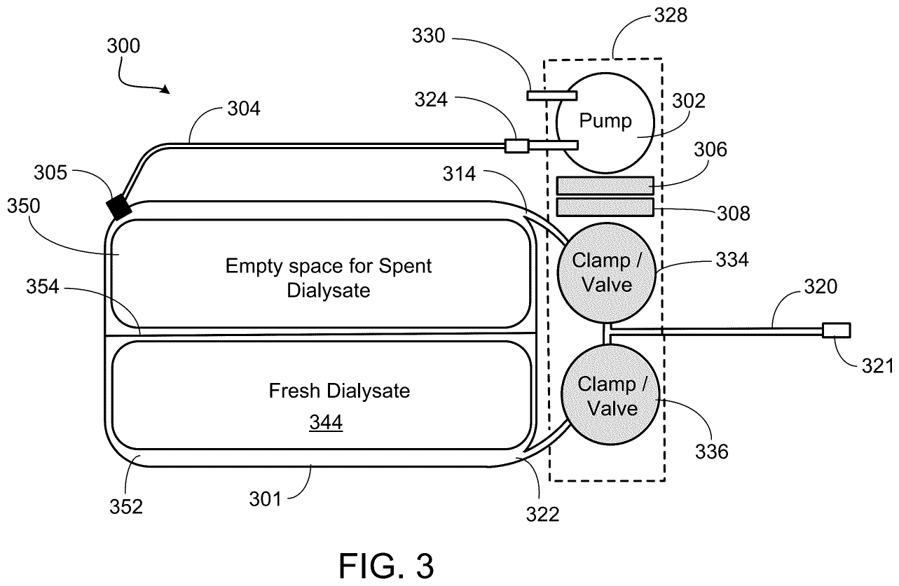

While the example PD treatment systems 100 and 200 have been depicted as having a separate drain bag and dialysate bag, in some implementations, the system can include a bifurcated bag that integrates the functionalities of both a fresh dialysate bag and a drain bag. FIG. 3 is a top-down view of a PD system 300 that includes a microfluidic pump 302 and a dual chamber bag 301. The microfluidic pump 302 is fluidically connected to the dual chamber bag 301 by a connector line 304.

The dual chamber bag 301 is made from a flexible material (e.g., a flexible plastic). The dual chamber bag 301 includes an effluent chamber 350 having an interior configured to collect effluent drained from a patient during a drain phase of a PD treatment, a dialysate chamber 352 configured to contain fresh dialysate 344 for the fill phase of a PD treatment, and a flexible membrane 354 separating the effluent chamber 350 from the dialysate chamber 352. The flexible membrane 354 is sealed to prevent contamination of fluid between the effluent chamber 350 and the dialysate chamber 352. The dual chamber design of the dual chamber bag 301 provides a compact and disposable solution that increases the efficiency, cleanliness, and portability of performing PD treatment.

The microfluidic pump 302 includes a casing 328. The casing 328 is configured to house electrical components 306 and a power source 308 (e.g., one or more batteries). For example, the electrical components 306 of the casing 328 can include a controller and at least one processor which can be used to record, store, and wirelessly transmit one or more parameters related to the PD treatment.

For example, the casing 328 houses a controller and at least one processor which can be used to record, store, and wirelessly transmit one or more parameters related to the PD treatment, other embodiments are possible. For example, a controller, in conjunction with at least one processor, is configured to execute the operation of the microfluidic pump 302, the valves (e.g., valve 324, transfer set valve 321, inlet valve 334, and outlet valve 336). In some implementations, the processor executes instructions from the controller to manage the flow rate and direction of flow of fresh and effluent. Further, the processor executes instructions from the controller to manage the flow rate and direction of flow of fluid (e.g., positive air pressure or negative air pressure) generated by the microfluidic pump 302 to the delivery of fresh and effluent as part of the PD treatment.

The microfluidic pump 302 is a piezoelectric disc pump. The microfluidic pump 302 is fluidly coupled to a valve 324. When an electric current is applied to the microfluidic pump 302, the disc of the piezoelectric disc pump changes shape (e.g., vibrates) due to a piezoelectric effect. The frequency of these vibrations can be in the ultrasonic range. The piezoelectric disc pump creates a positive pressure by vibrating at a frequency that causes the disc to move air out of a casing 328 through the connector line 304 and to the interior of the dual chamber bag 301. The direction of the fluid (e.g., air) movement can be controlled by the valve 324. To create a vacuum within the dual chamber bag 301, the piezoelectric disc pump draws fluid into the casing 328 from the interior of the effluent chamber 350 of the dual chamber bag 301 and expels the fluid via the fluid outlet 330. For example, by adjusting the vibration pattern or frequency of the piezoelectric disc, the microfluidic pump 302 is configured to pull fluid (e.g., air) into the casing 328, generating a negative pressure inside the effluent chamber 350 of the dual chamber bag 301 thereby generating a vacuum.

The connection of the connector line 304 to the interior of the dual chamber bag 301 produces an airtight seal, which enables the microfluidic pump 302 to provide a controlled positive or negative pressure to the dual chamber bag 301. For example, the connection of the connector line 304 and the dual chamber bag 301 is a luer-lock connector 305 comprising two parts: a male part (the plug) with a conical or tapered shape, and a female part (the socket), which has a corresponding conical or tapered cavity.

The effluent chamber 350 is fluidly connected to the transfer set 320 via an inlet line 314. The transfer set 320 is configured to be fluidly coupled to the peritoneal cavity of the patient. For example, the transfer set 320 is coupled to a transfer set valve 321 that is configured to couple to a transfer set of the patient and selectively permit or prevent medical fluid flow to or from the peritoneal cavity of the patient through the transfer set 320.

The inlet line 314 can include an inlet valve 334. The transfer set 320 is positioned is fluidly connected to the peritoneal cavity of the patient such that effluent can drain from the peritoneal cavity of the patient to the effluent chamber 350 via the transfer set 320 and the inlet line 314. For example, the inlet valve 334 is configured to permit effluent from the transfer set 320 to enter the effluent chamber 350 through the inlet line 314 but prevent the effluent from entering the transfer set 320 from the effluent chamber 350.

The dialysate chamber 352 is fluidly connected to the transfer set 320 via an outlet line 322. An outlet valve 336 is positioned along the outlet line 322 to control fresh dialysate 344 flow from the dialysate chamber 352 along the outlet line 322.

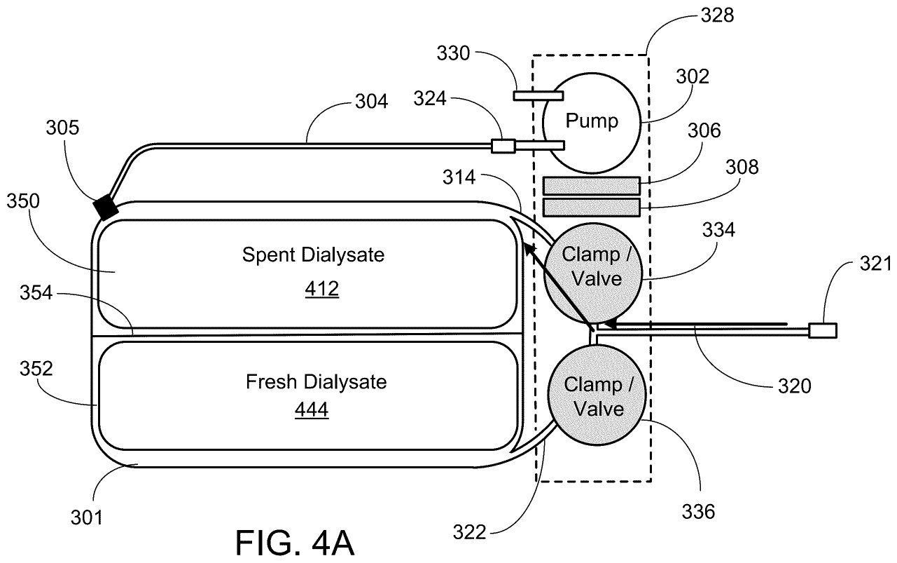

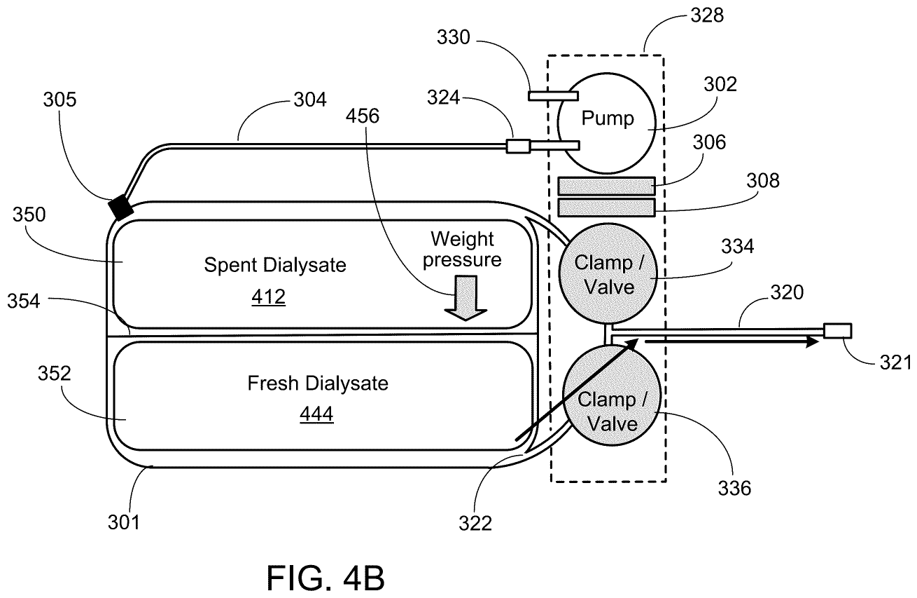

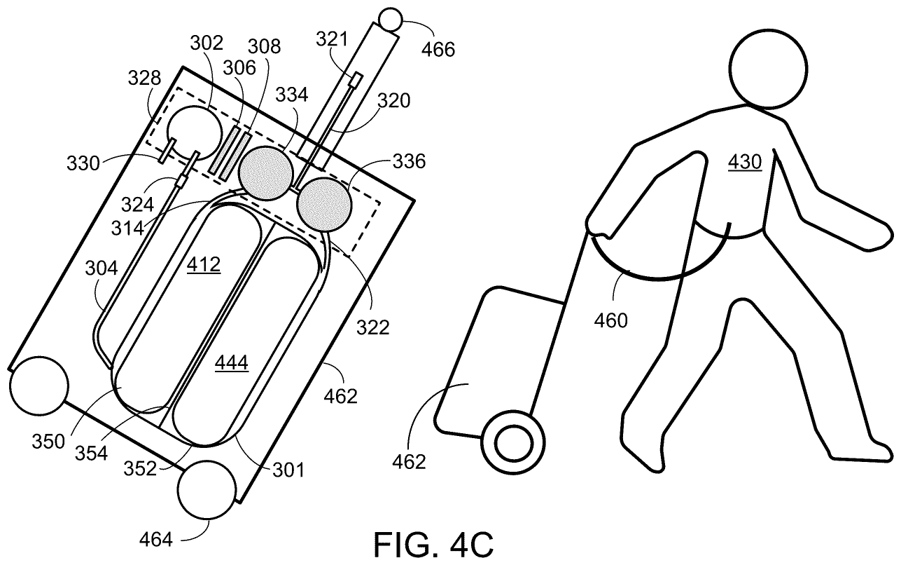

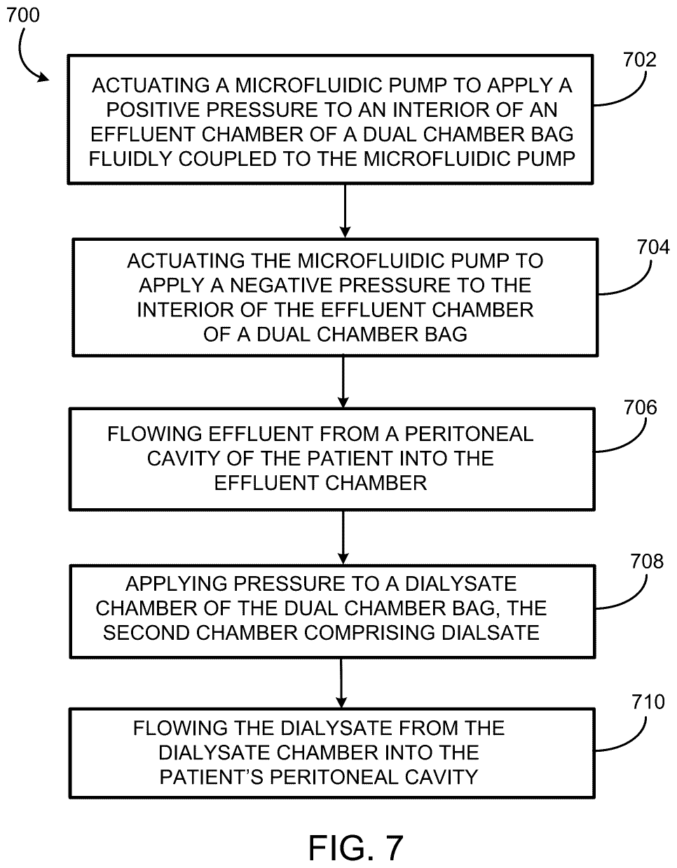

An example process of performing a drain phase of PD treatment and a fill phase of a PD treatment using the example system 300 will be described with reference to FIGS. 4A-4C, and 7. FIGS. 4A-4C depict an example process of performing a drain phase of PD treatment and a fill phase of a PD treatment using the dual chamber bag 301 and the components used in the example PD system 300. FIG. 7 depicts example method 700 for performing PD treatment using the dual chamber bag (e.g., the dual chamber bag 301).

The microfluidic pump 302 is actuated to apply a positive pressure to the interior of an effluent chamber 350 of the dual chamber bag 301 via a connector line 304 fluidly coupled to the microfluidic pump 302 and the dual chamber bag 301 (702). When an electric current is applied to the microfluidic pump 302, the disc of the piezoelectric disc pump changes shape (e.g., vibrates) due to a piezoelectric effect. The frequency of these vibrations can be in the ultrasonic range. The piezoelectric disc pump creates a positive pressure by vibrating at a frequency that causes the disc to move air out of a casing 328 through the connector line 304 and to the interior of the dual chamber bag 301. At The valves 334, 336 along the inlet line 314 and the outlet line 322, respectively, are closed while the microfluidic pump 302 actuates to apply a positive pressure to the interior of the effluent chamber 350. For example, the microfluidic pump 302 pumps fluid (e.g., air) through the connector line 304 and into the effluent chamber 350 of the dual chamber bag 301, which generates a positive pressure within the effluent chamber 350. Filling the interior volume of the effluent chamber 350 with air using the microfluidic pump 302 prepares the effluent chamber 350 to collect effluent 412 at the end of a dwell period by preventing the effluent chamber 350 from collapsing when negative pressure is applied to the effluent chamber 350 during the drain phase, as will be described in further detail herein. The inlet valve 334 prevents the air pumped into the effluent chamber 350 by the microfluidic pump 302 from entering the transfer set 320.

Turning to the embodiment of FIG. 4A, at the end of a dwell period of the PD treatment, the valve 334 along the inlet line 314 is opened and the microfluidic pump 302 is actuated to apply a negative pressure to the interior of the effluent chamber 350 of the dual chamber bag 301 (704) by drawing the positive pressure fluid from the interior of the effluent chamber 350 into the casing 328 of and exiting via the fluid outlet 330. For example, the microfluidic pump 302 operates to draw fluid (e.g., air) through the connector line 304.

The negative pressure generated by the microfluidic pump 302 produces a vacuum in the interior of the effluent chamber 350 that causes effluent 412 from the patient's peritoneal cavity to flow through the transfer set valve 321, through the transfer set 320, through the inlet line 314, and into the effluent chamber 350 (706).

Once the patient's peritoneal cavity has been drained of effluent 412, the fill phase of the PD treatment can be performed. FIG. 4B shows an example fill phase with the dialysate chamber 352 containing fresh dialysate 444 and positioned adjacent the effluent chamber 350. The flexible membrane 354 separates the effluent 412 from the fresh dialysate 444, which prevent contamination of the dialysate 444 contained within the dialysate chamber 352. To perform a fill phase of the PD treatment using the dual chamber bag 301, the valve 334 along the inlet line 314 is closed and the outlet valve 336 along the outlet line 322 is opened. The weight of effluent 412 contained within the effluent chamber 350 from the drain phase applies a positive pressure the flexible membrane 354, which flexes and transfers the pressure to the dialysate chamber 352 as indicated by the arrow 456. The weight of the effluent chamber 350 (filled with effluent 412) thereby applies a positive pressure (e.g., squeeze) to an internal portion of the dialysate chamber 352 (708). As a result of the positive pressure applied to the internal portion of the dialysate chamber 352, fresh dialysate 444 is caused to flow through the outlet line 322 and through the transfer set 320 to deliver the dialysate 444 contained within the dialysate chamber 352 to the patient's peritoneal cavity (710). In some implementations, the dialysate chamber 352 is configured to contain about 1-3 liters of fresh dialysate 444.

FIG. 4C shows an example of the dual chamber bag 301 encased in a rigid container for portability. The dual chamber bag 301 is positioned inside of a rigid case 462. The rigid container is configured to allow portability of the PD treatment system 300 and includes wheels 464, a handle 466, and an extended transfer set 460. In this way, a patient 430 has increased flexibility as they can administer a PD treatment while in moving about their daily lives.

In some example embodiments, the fresh dialysate (e.g., 244, 344, and 444) is heated prior to a fill phase of a PD treatment. For example, the rigid container (e.g., 246) or rigid case (e.g., 462) can include a heating element to heat the fresh dialysate to approximately body temperature prior to a fill phase of a PD treatment.

While the fluid line connectors 105, 203, 205, and 305 have been described as a luer-lock connectors, in some implementations the connection can be a barbed fitting. A barbed fitting is a stem with one or more barbs or ridges. The tubing (e.g., the fluid line) is stretched over the barbed end, and the elasticity of the tubing material causes it to tighten around the barbs. The barbs are angled back towards the base of the fitting, making it easy to push the tubing onto the fitting but difficult to pull it off, as the barbs act against the direction of removal.

While drain bag 110, 210 has been described as being connected to the inlet line 114, 214 prior to the drain bag 110, 210 being filled with positive pressure, in some implementations, the drain bag 110, 210 is connected to the inlet line 114, 214 after the drain bag 110, 210 is filled with fluid from the positive pressure generated by the microfluidic pump 102.

While the flow sensor 118, 218 has been described as generating an audio alert, in some implementations this alert could be in the form of a visual signal or a notification on a connected device, prompting the patient to check the system for any issues or to adjust the treatment as necessary.

Additionally, FIG. 3 describes a valve 324, in some implementations, the valve is a solenoid valve. FIG. 3 further describes the connector line 304 as including the valve 324, in some implementations the connector line 304 can also include a pressure sensor (e.g., pressure sensor 126 or 226).

While FIG. 2C depicts that a constant pressure is applied to the rigid container, in some implementations the pressure applied to the rigid container 246 would be increased over time as the dialysate bag 208 became less full of fresh dialysate 244.

While FIG. 1 depicts a drain bag 110 that is coupled to a drain line 116, in some implementations the drain bag 110 is not connected to a drain line and the effluent in the drain bag is manually disposed of by the patient (e.g., by pouring the content of the drain bag down a bathtub train or into a toilet).

While FIGS. 1-4C describe the valves (e.g., solenoid valve 124, transfer set valve 121, inlet valve 134, and outlet valve 136, solenoid valve 224, transfer set valve 221, inlet valve 234, and outlet valve 236) as operated with a controller, in some implementations the valves are operated manually. For example, the valves can be operated by prompting a user to open and close the valves (e.g., with printed instructions, link smartphone to monitor the process, audible prompts, etc.).

The pressure sensor 226 is continuously monitored by the processor, which analyzes the pressure data to ensure that the fluid pressure within the system remains within predefined limits. Should the pressure deviate from these limits, the controller can make real-time adjustments to the operation of the microfluidic pump 202 and the solenoid valve 224, either by altering the pump's speed or by adjusting the valve's position, to bring the system back to the desired operational parameters. This monitoring and adjustment capability is essential for preventing complications during the PD treatment by maintaining optimal pressure conditions within the system.

Moreover, the electrical components within the casing 228 are designed to ensure efficient power management and reliable communication between the controller, the processor, and the various components of the system. The controller can also wirelessly transmit data related to the operation of the microfluidic pump 202, solenoid valve 224, and pressure sensor 226 to an external device, allowing for remote monitoring and adjustment of the PD treatment by healthcare professionals. This capability enhances the safety and effectiveness of the treatment, providing real-time insights into the treatment process and enabling prompt interventions when necessary.

What is claimed is:

1. A system for draining fluid from a peritoneal cavity of a patient comprising:
a microfluidic pump;
a drain bag fluidly coupled to the microfluidic pump; and
an inlet line coupled to the drain bag, wherein the microfluidic pump is configured to apply a negative pressure to an interior of the drain bag to draw effluent from a peritoneal cavity of the patient along the inlet line and into the drain bag.

2. The system of claim 1, wherein the microfluidic pump is a piezoelectric pump.

3. The system of claim 1, further comprising:
a drain line coupled to the drain bag; and
a flow sensor coupled to the drain line, wherein the flow sensor is configured to measure a volume a fluid flowing from the drain bag along the drain line.

4. The system of claim 1, wherein the microfluidic pump is configured to apply a positive pressure to the interior of the drain bag prior to applying the negative pressure to the interior of the drain bag.

5. The system of claim 1, further comprising a connector line fluidly coupled to the microfluidic pump and the drain bag.

6. The system of claim 5, further comprising:
a solenoid valve coupled to the connector line and configured to control a flow of fluid generated by the microfluidic pump to the drain bag or away from the drain bag; and
a pressure sensor coupled to the connector line, wherein the pressure sensor is configured to detect a pressure generated by the microfluidic pump and applied to the interior of the drain bag.

7. The system of claim 1, further comprising a casing surrounding the microfluid pump.

8. The system of claim 7, further comprising a control unit positioned within the casing, the control unit comprising at least one processor configured to control operations of the microfluidic pump.

9. The system of claim 1, further comprising a valve coupled to the inlet line, wherein the valve is configured to control flow of spent medical fluid into the drain bag.

10. The system of claim 1, further comprising a rigid container fluidly coupled to the microfluidic pump.

11. The system of claim 10, further comprising a dialysate bag positioned inside the rigid container.

12. A method for performing peritoneal dialysis, the method comprising:
actuating a microfluidic pump to apply a positive pressure to an interior of a drain bag; and
actuating the microfluidic pump to apply a negative pressure to the interior of the drain bag to generate a vacuum that causes effluent to flow from a peritoneal cavity of a patient into the drain bag.

13. The method of claim 12, further comprising:
flowing effluent from the interior of the drain bag along a drain line coupled to the drain bag; and
measuring, with a flow sensor, a volume of the effluent flowed from the drain bag along the drain line with the flow sensor.

14. The method of claim 12, further comprising:
actuating the microfluidic pump to apply the positive pressure to a dialysate bag positioned inside an interior of a rigid container fluidly coupled to the microfluidic pump; and
opening a valve along a second fluid line fluidly coupling the dialysate bag and the peritoneal cavity of a patient, wherein a pressure applied to the dialysate bag causes fresh dialysate to flow from the dialysate bag to the patient's peritoneal cavity along the second fluid line.

15. The method of claim 12, wherein the microfluidic pump is a piezoelectric pump and (a) further comprises applying an electric current to the piezoelectric pump generating a vibration at a first frequency, wherein the first frequency causes air movement from the piezoelectric pump and into the interior of the drain bag.

16. The method of claim 15, further comprises applying another electric current to the piezoelectric pump generating another vibration at a second frequency, wherein the second frequency causes air movement from the interior of the drain bag and into the piezoelectric pump.

17. The method of claim 12, the method further comprising detecting, with a pressure sensor, the positive pressure of the interior of the drain bag.

18. The method of claim 12, the method further comprising detecting, with a pressure sensor, the negative pressure of the interior of the drain bag.

19. A system for administering and draining fluid from a peritoneal cavity of a patient comprising:
a microfluidic pump;
a drain bag fluidly coupled to the microfluidic pump,
a rigid container fluidly coupled to the microfluidic pump; and
a dialysate bag positioned inside the rigid container.

20. The system of claim 19, wherein the microfluidic pump is a piezoelectric pump.

21. The system of claim 19, further comprising:
an inlet line configured to fluidly couple the drain bag to the peritoneal cavity of the patient; and
an outlet line configured to fluidly couple the dialysate bag to the peritoneal cavity of the patient.

22. The system of claim 21, further comprising:
an inlet valve coupled to the inlet line and configured to control flow of effluent the inlet line; and
an outlet valve coupled to the outlet line and configured to control flow of fresh dialysate along the outlet line.

23. The system of claim 19, further comprising:
a connector line fluidly coupled to the microfluidic pump, the drain bag, and the rigid container;
a solenoid valve coupled to the connector line configured to control a flow of fluid generated by the microfluidic pump along the connector line; and
a pressure sensor coupled to the connector line and configured to detect a pressure generated by the microfluidic pump.

24. The system of claim 19, further comprising:
a first fluid valve coupled to a first fluid line, and configured to control a flow of fluid generated by the microfluidic pump through the first fluid line; and
a second fluid valve coupled to a second fluid line and configured to control the flow of fluid generated by the microfluidic pump through the second fluid line.

25. The system of claim 19, further comprising:
a connector line coupled to the microfluidic pump;
a T-connector coupled to the connector line;
a first fluid line coupled to the T-connector; and
a second fluid line coupled to the T-connector, wherein the first fluid line and the second fluid line selectively guide a fluid generated by the microfluidic pump.

26. The system of claim 19, wherein the microfluidic pump is configured to apply a negative pressure to an interior of the drain bag to draw spent medical fluid from the patient to the interior of the drain bag.

27. The system of claim 19, wherein the microfluidic pump is configured to apply a positive pressure to an interior of the rigid container to thereby affect a pressure to an external portion of the dialysate bag.

28. A method of performing peritoneal dialysis, the method comprising:
actuating a microfluidic pump to apply a positive pressure to an interior of a drain bag fluidly coupled to the microfluidic pump;
actuating the microfluidic pump to apply a negative pressure to the interior of the drain bag to generate a vacuum inside the drain bag;
opening a first valve along a first fluid line fluidly coupling the drain bag and a peritoneal cavity of a patient, wherein the vacuum generated inside the drain bag causes effluent to flow from the patient's peritoneal cavity to the drain bag;
actuating the microfluidic pump to apply the positive pressure to a dialysate bag positioned inside an interior of a rigid container fluidly coupled to the microfluidic pump; and
opening a second valve along a second fluid line fluidly coupling the dialysate bag and the peritoneal cavity of the patient, wherein the positive pressure applied to the dialysate bag causes fresh dialysate to flow from the dialysate bag to the patient's peritoneal cavity along the second fluid line.

29. The method of claim 28, the method further comprising:
flowing effluent from the drain bag along a drain line coupled to the drain bag; and
measuring, with a flow sensor, a volume of the effluent flowing from the drain bag along the drain line with the flow sensor.

30. The method of claim 28, wherein the microfluidic pump is a piezoelectric pump and actuating the microfluidic pump to apply the positive pressure to an interior of the drain bag comprises applying an electric current to the piezoelectric pump to generate a vibration at a first frequency, wherein the first frequency causes air movement from the piezoelectric pump into the interior of the drain bag.

31. The method of claim 30, wherein actuating the microfluidic pump to apply the negative pressure to the interior of the drain bag comprises applying a second electric current to the piezoelectric pump to generate a second vibration at a second frequency, wherein the second frequency causes air movement from the interior of the drain bag into the piezoelectric pump.

32. A system for performing peritoneal dialysis, the system comprising:
a microfluidic pump;
a dual chamber bag fluidly coupled to the microfluidic pump, the dual chamber bag comprising:
an effluent chamber configured to be fluidly coupled to a peritoneal cavity of a patient and receive effluent from the peritoneal cavity of the patient;
a dialysate chamber configured to be fluidly coupled to the peritoneal cavity of a patient and to contain dialysate; and
a flexible membrane separating the effluent chamber from the dialysate chamber.

33. The system of claim 32, wherein the microfluidic pump is a piezoelectric pump.

34. The system of claim 32, wherein the microfluidic pump is configured to apply a negative pressure to an interior of the effluent chamber to draw spent medical fluid from the patient to the interior of the effluent chamber.

35. The system of claim 32, wherein the flexible membrane is configured to flex to apply a pressure to the dialysate chamber when the effluent chamber is filled with effluent.

36. The system of claim 35, wherein the pressure to the dialysate chamber is from a weight of the effluent contained in the effluent chamber.

37. The system of claim 32, further comprising:
an inlet line coupled to the effluent chamber;
an inlet valve coupled to the inlet line, wherein the inlet valve is configured to control flow of effluent into the effluent chamber from the peritoneal cavity of the patient;
an outlet line coupled to the dialysate chamber; and
an outlet valve coupled to the outlet line, wherein the outlet valve is configured to control flow of dialysate from the dialysate chamber to the peritoneal cavity of the patient.

38. The system of claim 37, further comprising a rigid case configured to contain the microfluidic pump and the dual chamber bag.

39. The system of claim 38, wherein the rigid case comprises a heater configured to heat dialysate contained in the dialysate chamber.

40. The system of claim 38, wherein the rigid case further comprises a handle and wheels.

41. A method of performing peritoneal dialysis treatment, the method comprising:
actuating a microfluidic pump to apply a positive pressure to an interior of an effluent chamber of a dual chamber bag fluidly coupled to the microfluidic pump;
actuating the microfluidic pump to apply a negative pressure to the interior of the effluent chamber of the dual chamber bag, wherein the negative pressure in the interior of the effluent chamber creates a vacuum inside the effluent chamber;
flowing effluent from a peritoneal cavity of a patient into the effluent chamber;
applying pressure to a dialysate chamber of the dual chamber bag, the dialysate chamber comprising dialysate; and
flowing the dialysate from the dialysate chamber into the patient's peritoneal cavity.

42. The method of claim 41, wherein flowing effluent from the peritoneal cavity of the patient into the effluent chamber comprises opening a valve along an inlet line fluidly coupling the effluent chamber to the peritoneal cavity.

43. The method of claim 41, wherein flowing the dialysate from the dialysate chamber into the patient's peritoneal cavity comprises opening a valve along an outlet line fluidly coupling the effluent chamber to the peritoneal cavity.

44. The method of claim 41, further comprising:
positioning the dual chamber bag inside a rigid case comprising a handle and one or more wheels; and
transporting the dual chamber bag during the treatment.
